(19) United States
(12) Patent Application Publication
(10) Pub. No.: US 2024/0398361 A1
(43) Pub. Date: Dec. 5, 2024

(54) SYSTEMS AND METHODS FOR PARAMETRIC IMAGING IN POSITRON EMISSION TOMOGRAPHY

(71) Applicant: SHANGHAI UNITED IMAGING HEALTHCARE CO., LTD., Shanghai (CN)

(72) Inventors: Qing YE, Shanghai (CN); Yihuan LU, Shanghai (CN)

(73) Assignee: SHANGHAI UNITED IMAGING HEALTHCARE CO., LTD., Shanghai (CN)

(21) Appl. No.: 18/676,697

(22) Filed: May 29, 2024

(30) Foreign Application Priority Data
May 29, 2023 (CN) 202310620627.6

(51) Int. Cl. A61B 6/03; A61B 6/00; G06T 7/11

(52) U.S. Cl. CPC A61B 6/037; A61B 6/5205; A61B 6/5258; G06T 7/11; G06T 2207/10104; G06T 2207/30096

(57) ABSTRACT

A method and a system for parametric imaging in PET may be provided. Multiple PET images collected via a PET scan of a target subject may be obtained. The PET images may be dynamic PET images for difference time periods during the PET scan and used to generate a target PET parametric image of the target subject. Whether data correction needs to be performed in a process of generating the target PET parametric image may be determined. In response to determining that data correction needs to be performed, the PET images or a preliminary PET parametric image may be corrected, and the target PET parametric image of the target subject may be generated based on the corrected PET images or the corrected preliminary PET parametric image, wherein the preliminary PET parametric image is generated by processing the PET images using at least one pharmacokinetic model.

### CROSS-REFERENCE TO RELATED APPLICATIONS

This application claims priority of Chinese Patent Application No. 202310620627.6 filed on May 29, 2023, the contents of which are incorporated herein by reference.

### TECHNICAL FIELD

The present disclosure relates to positron emission tomography (PET) imaging, and in particular, to parametric imaging in PET.

### BACKGROUND

PET imaging has been widely used in clinical examination and disease diagnosis in recent years. Parametric imaging technique in PET can provide a quantitative measurement result having a high accuracy. For example, the parametric imaging technique can provide voxel-level dynamics of a tracer uptake by applying kinetic modeling for each individual voxel.

### SUMMARY

According to an aspect of the present disclosure, a method for parametric imaging in PET may be provided. The method may be implemented on a computing device having at least one processor and at least one storage device. The method may include obtaining multiple PET images collected via a PET scan of a target subject. The PET images may be dynamic PET images for difference time periods during the PET scan and used to generate a target PET parametric image of the target subject. The method may also include determining whether data correction needs to be performed in a process of generating the target PET parametric image. The method may further include, in response to determining that data correction needs to be performed, correcting the PET images or a preliminary PET parametric image, and generating the target PET parametric image of the target subject based on the corrected PET images or the corrected preliminary PET parametric image, wherein the preliminary PET parametric image is generated by processing the corrected PET images or the PET images using at least one pharmacokinetic model.

In some embodiments, to determine whether data correction needs to be performed in a process of generating the target PET parametric image, the method may include the following operations. The method may include, for each of the PET images, generating an organ segmentation image by segmenting organs in the PET image. The method may also include determining motion information of the organs of the target subject based on the organ segmentation image corresponding to each of the PET images. The method may further include determining whether motion correction needs to be performed in the process of generating the target PET parametric based on the motion information of the organs.

In some embodiments, to determine whether data correction needs to be performed in a process of generating the target PET parametric image, the method may include the following operations. The method may include, for each of the PET images, determining a target region corresponding to a target organ in the PET image, and determining a variation of pixel values in the target region. The method may further include determining whether noise correction needs to be performed in the process of generating the target PET parametric image based on the variation corresponding to each of the PET images.

In some embodiments, to determine whether data correction needs to be performed in a process of generating the target PET parametric image, the method may include the following operations. The method may include, for each physical point of the target subject, determining a pharmacokinetic model corresponding to the physical point, and determining a PET parametric value of the physical point based on pixel values corresponding to the physical point in the corrected PET images or the PET images using the pharmacokinetic model of the physical point. The method may also include generating the preliminary PET parametric image based on the PET parametric value of each physical point of the target subject. The method may further include determining whether data correction needs to be performed in the process of generating the target PET parametric image based on the preliminary PET parametric image.

In some embodiments, the preliminary PET parametric image may include a first preliminary PET parametric image and a second preliminary PET parametric image corresponding to different PET parameters, and to determine whether data correction needs to be performed in the process of generating the target PET parametric image based on the preliminary PET parametric image, the method may include the following operations. The method may include determining a first lesion segmentation result by segmenting lesion areas from the first preliminary PET parametric image. The method may also include determining a second lesion segmentation result by segmenting lesion areas from the second preliminary PET parametric image. The method may also include determining a similarity between the first lesion segmentation result and the second lesion segmentation result. The method may further include determining whether motion correction needs to be performed in the process of generating the target PET parametric image based on the similarity.

In some embodiments, to determine whether data correction needs to be performed in a process of generating the target PET parametric image, the method may include the following operations. The method may include, for each physical point of the target subject, determining a time-activity curve (TAC) of the physical point based on pixel values corresponding to the physical point in the PET images, and determining a noise level parameter corresponding to the physical point based on the TAC of the physical point. The method may further include determining whether noise correction needs to be performed in a process of generating the target PET parametric image based on the noise level parameter corresponding to each physical point.

In some embodiments, to determine whether data correction needs to be performed in the process of generating the target PET parametric image based on the preliminary PET parametric image, the method may include the following operations. The method may include, for each physical point of the target subject, determining a matching degree of the pharmacokinetic model with respect to the physical point based on the corrected PET images or the PET images, and generating a determination result indicating whether the corresponding pixel of the physical point in the preliminary PET parametric image needs to be corrected based on the matching degree. The method may further include determining whether pharmacokinetic model correction needs to be performed in the process of generating the target PET parametric image based on the determination result corresponding to each physical point.

In some embodiments, to determine a pharmacokinetic model corresponding to the physical point, the method may include the following operations. The method may include determining a TAC of the physical point based on pixel values corresponding to the physical point in the PET images. The method may further include selecting, from multiple candidate pharmacokinetic models, the pharmacokinetic model corresponding to the physical point based on the TAC of the physical point.

In some embodiments, the pharmacokinetic model corresponding to the physical point may be determined by processing the TAC of the physical point using a selection model. The selection model may be generated by training a preliminary model using training samples. Each of the training samples may correspond to a sample physical point and may be determined by performing the following operations. A sample TAC of the sample physical point may be determined based on sample PET images collected in a sample PET scan of a sample subject. Predicted tacs of the sample physical point corresponding to the candidate pharmacokinetic models may be determined by processing the sample PET images using the candidate pharmacokinetic models. A recommended pharmacokinetic model may be determined from the candidate pharmacokinetic models based on the sample TAC and the predicted tacs. The sample TAC may be designated as a training input of the training sample and the recommended pharmacokinetic model may be designated as a training label of the training sample.

In some embodiments, to determine whether data correction needs to be performed in a process of generating the target PET parametric image, the method may include the following operations. The method may include obtaining sets of PET raw data collected in the PET scan. The PET images may be reconstructed from the sets of PET raw data. The method may include, for each set of PET raw data, generating a histoimage corresponding to the set of PET raw data. The method may also include determining motion information of organs of the target subject based on the histoimage corresponding to each set of PET raw data. The method may further include determining whether motion correction needs to be performed in the process of generating the target PET parametric image based on the motion information of the organs.

In some embodiments, to determine whether data correction needs to be performed in a process of generating the target PET parametric image, the method may include the following operations. The method may include obtaining sets of PET raw data collected in the PET scan. The PET images may be reconstructed from the sets of PET raw data. The method may include, for each set of PET raw data, determining a coincidence event count based on the set of PET raw data, and determining a noise level parameter of the set of PET raw data based on the coincidence event count corresponding to the set of PET raw data. The method may further include determining whether noise correction needs to be performed in the process of generating the target PET parametric image based on the noise level parameter of each set of PET raw data.

In some embodiments, to obtain multiple PET images collected in a PET scan of the target subject, the method may include the following operations. The method may include obtaining multiple sets of PET raw data collected in the PET scan. The method may also include, for each set of PET raw data, determining coincidence event count based on the set of PET raw data, and determining a reconstruction algorithm for reconstructing the set of PET raw data based on the coincidence event count. The method may further include generating the PET images by reconstructing the sets of PET raw data using their respective reconstruction algorithms.

In some embodiments, to determine whether data correction needs to be performed in a process of generating the target PET parametric image, the method may include the following operations. The method may include performing a first quality evaluation on the PET images. The method may also include performing a second quality evaluation on the preliminary PET parametric image. The method may further include determining whether data correction needs to be performed in the process of generating the target PET parametric image based on results of the first quality evaluation and the second quality evaluation.

According to another aspect of the present disclosure, a system for parametric imaging in PET may be provided. The system may include at least one storage device including a set of instructions and at least one processor. The at least one processor may be configured to communicate with the at least one storage device. When executing the set of instructions, the at least one processor may be configured to direct the system to perform one or more of the following operations. The system may obtain multiple PET images collected via a PET scan of a target subject. The PET images may be dynamic PET images for difference time periods during the PET scan and used to generate a target PET parametric image of the target subject. The system may also determine whether data correction needs to be performed in a process of generating the target PET parametric image. The system may obtain, in response to determining that data correction needs to be performed, correct the PET images or a preliminary PET parametric image, and generate the target PET parametric image of the target subject based on the corrected PET images or the corrected preliminary PET parametric image, wherein the preliminary PET parametric image is generated by processing the PET images using at least one pharmacokinetic model.

According to yet another aspect of the present disclosure, a non-transitory computer readable medium may be provided. The non-transitory computer readable medium may include at least one set of instructions for parametric imaging in PET. When executed by one or more processors of a computing device, the at least one set of instructions may cause the computing device to perform a method. The method may include obtaining multiple PET images collected via a PET scan of a target subject. The PET images may be dynamic PET images for difference time periods during the PET scan and used to generate a target PET parametric image of the target subject. The method may also include determining whether data correction needs to be performed in a process of generating the target PET parametric image. The method may further include, in response to determining that data correction needs to be performed, correcting the PET images or a preliminary PET parametric image, and generating the target PET parametric image of the target subject based on the corrected PET images or the corrected preliminary PET parametric image, wherein the preliminary PET parametric image is generated by processing the PET images using at least one pharmacokinetic model.

Additional features will be set forth in part in the description which follows, and in part will become apparent to those skilled in the art upon examination of the following and the accompanying drawings or may be learned by production or operation of the examples. The features of the present disclosure may be realized and attained by practice or use of various aspects of the methodologies, instrumentalities, and combinations set forth in the detailed examples discussed below.

### BRIEF DESCRIPTION OF THE DRAWINGS

The present disclosure is further described in terms of exemplary embodiments. These exemplary embodiments are described in detail with reference to the drawings. These embodiments are non-limiting exemplary embodiments, in which like reference numerals represent similar structures throughout the several views of the drawings, and wherein:

FIG. 1 is a schematic diagram illustrating an exemplary PET system according to some embodiments of the present disclosure;

FIG. 2 is a schematic diagram illustrating exemplary hardware and/or software components of an exemplary computing device according to some embodiments of the present disclosure;

FIG. 3 is a block diagram illustrating an exemplary processing device according to some embodiments of the present disclosure;

FIG. 4A is a flowchart illustrating an exemplary process for parametric imaging in PET according to some embodiments of the present disclosure;

FIG. 4B is a schematic diagram illustrating an exemplary process for determining whether data correction is needed according to some embodiments of the present disclosure;

FIG. 5 is a flowchart illustrating an exemplary process for determining whether motion correction is needed according to some embodiments of the present disclosure;

FIG. 6 is a flowchart illustrating an exemplary process for determining whether noise correction is needed according to some embodiments of the present disclosure;

FIG. 7 is a schematic diagram illustrating an exemplary process for determining a pharmacokinetic model corresponding to a physical point using a selection mode according to some embodiments of the present disclosure;

FIG. 8 is a flowchart illustrating an exemplary process for determining whether motion correction is needed according to some embodiments of the present disclosure;

FIG. 9 is a flowchart illustrating an exemplary process for determining whether noise correction is needed according to some embodiments of the present disclosure; and

FIG. 10 is a flowchart illustrating an exemplary process for determining whether pharmacokinetic model correction is needed according to some embodiments of the present disclosure.

### DETAILED DESCRIPTION

In the following detailed description, numerous specific details are set forth by way of examples in order to provide a thorough understanding of the relevant disclosure. However, it should be apparent to those skilled in the art that the present disclosure may be practiced without such details. In other instances, well-known methods, procedures, systems, components, and/or circuitry have been described at a relatively high level, without detail, in order to avoid unnecessarily obscuring aspects of the present disclosure. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown, but to be accorded the widest scope consistent with the claims.

In the following detailed description, numerous specific details are set forth by way of examples in order to provide a thorough understanding of the relevant disclosure. However, it should be apparent to those skilled in the art that the present disclosure may be practiced without such details. In other instances, well-known methods, procedures, systems, components, and/or circuitry have been described at a relatively high level, without detail, in order to avoid unnecessarily obscuring aspects of the present disclosure. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown, but to be accorded the widest scope consistent with the claims.

The terminology used herein is for the purpose of describing particular example embodiments only and is not intended to be limiting. As used herein, the singular forms “a,” “an,” and “the” may be intended to include the plural forms as well, unless the context clearly indicates otherwise. It will be further understood that the terms “comprise,” “comprises,” and/or “comprising,” “include,” “includes,” and/or “including,” when used in this specification, specify the presence of stated features, integers, steps, operations, elements, and/or components, but do not preclude the presence or addition of one or more other features, integers, steps, operations, elements, components, and/or groups thereof.

It will be understood that the term “system,” “engine,” “unit,” “module,” and/or “block” used herein are one method to distinguish different components, elements, parts, sections or assembly of different levels in ascending order. However, the terms may be displaced by another expression if they achieve the same purpose.

Generally, the word “module,” “unit,” or “block,” as used herein, refers to logic embodied in hardware or firmware, or to a collection of software instructions. A module, a unit, or a block described herein may be implemented as software and/or hardware and may be stored in any type of non-transitory computer-readable medium or another storage device. In some embodiments, a software module/unit/block may be compiled and linked into an executable program. It will be appreciated that software modules can be callable from other modules/units/blocks or from themselves, and/or may be invoked in response to detected events or interrupts. Software modules/units/blocks configured for execution on computing devices may be provided on a computer-readable medium, such as a compact disc, a digital video disc, a flash drive, a magnetic disc, or any other tangible medium, or as a digital download (and can be originally stored in a compressed or installable format that needs installation, decompression, or decryption prior to execution). Such software code may be stored, partially or fully, on a storage device of the executing computing device, for execution by the computing device. Software instructions may be embedded in firmware, such as an EPROM. It will be further appreciated that hardware modules/units/blocks may be included in connected logic components, such as gates and flip-flops, and/or can be included of programmable units, such as programmable gate arrays or processors. The modules/units/blocks or computing device functionality described herein may be implemented as software modules/units/blocks, but may be represented in hardware or firmware. In general, the modules/units/blocks described herein refer to logical modules/units/blocks that may be combined with other modules/units/blocks or divided into sub-modules/sub-units/sub-blocks despite their physical organization or storage. The description may be applicable to a system, an engine, or a portion thereof.

It will be understood that when a unit, engine, module, or block is referred to as being “on,” “connected to,” or “coupled to,” another unit, engine, module, or block, it may be directly on, connected or coupled to, or communicate with the other unit, engine, module, or block, or an intervening unit, engine, module, or block may be present, unless the context clearly indicates otherwise. As used herein, the term “and/or” includes any and all combinations of one or more of the associated listed items. The term “pixel” and “voxel” in the present disclosure are used interchangeably to refer to an element of an image.

These and other features, and characteristics of the present disclosure, as well as the methods of operation and functions of the related elements of structure and the combination of parts and economies of manufacture, may become more apparent upon consideration of the following description with reference to the accompanying drawings, all of which form a part of this disclosure. It is to be expressly understood, however, that the drawings are for the purpose of illustration and description only and are not intended to limit the scope of the present disclosure. It is understood that the drawings are not to scale.

In the present disclosure, a representation of a subject (e.g., an object, a patient, or a portion thereof) in an image may be referred to as “subject” for brevity. For instance, a representation of an organ, tissue (e.g., a heart, a liver, a lung), or an ROI in an image may be referred to as the organ, tissue, or ROI, for brevity. Further, an image including a representation of a subject, or a portion thereof, may be referred to as an image of the subject, or a portion thereof, or an image including the subject, or a portion thereof, for brevity. Still further, an operation performed on a representation of a subject, or a portion thereof, in an image may be referred to as an operation performed on the subject, or a portion thereof, for brevity. For instance, a segmentation of a portion of an image including a representation of an ROI from the image may be referred to as a segmentation of the ROI for brevity.

The parametric imaging technique usually needs a long scan time and a complex protocol. Since the parametric imaging is affected by many factors, it is possible to obtain PET data with relatively low accuracy. Conventionally, the original PET data (uncorrected data) is directly used to reconstruct a PET parametric image, which may result in artifacts, noise, etc., in the PET parametric image, thereby failing to meet the accuracy requirements for the PET parametric image. In this case, a new PET imaging needs to be performed to obtain a new PET parametric image that satisfies requirements. However, this conventional way has a low imaging efficiency and high imaging costs. Thus, it is desirable to develop methods and systems for parametric imaging, thereby improving the efficiency and accuracy of parametric imaging.

An aspect of the present disclosure relates to systems and methods for parametric imaging technique in PET. The systems may obtain multiple PET images collected via a PET scan of the target subject. The PET images may be used to generate a target PET parametric image of the target subject. The systems may determine whether data correction needs to be performed in a process of generating the target PET parametric image. In response to determining that data correction needs to be performed, the systems may correct the PET images or a preliminary PET parametric image, and generate the target PET parametric image of the target subject based on the corrected PET images or the corrected preliminary PET parametric image. The preliminary PET parametric image may be generated by processing the PET images using at least one pharmacokinetic model.

The methods and systems of the present disclosure may determine whether data correction needs to be performed in advance, automatically correct the PET images or the preliminary PET parametric image if needed, and generate the target PET parametric image based on the corrected PET images or the corrected preliminary PET parametric image. In this way, an accurate target PET parametric image can be obtained, the efficiency of the parametric imaging can be improved since no new PET imaging is required. The terms “automatic” and “automated” are used interchangeably referring to methods and systems that analyze information and generate results with little or no direct human intervention.

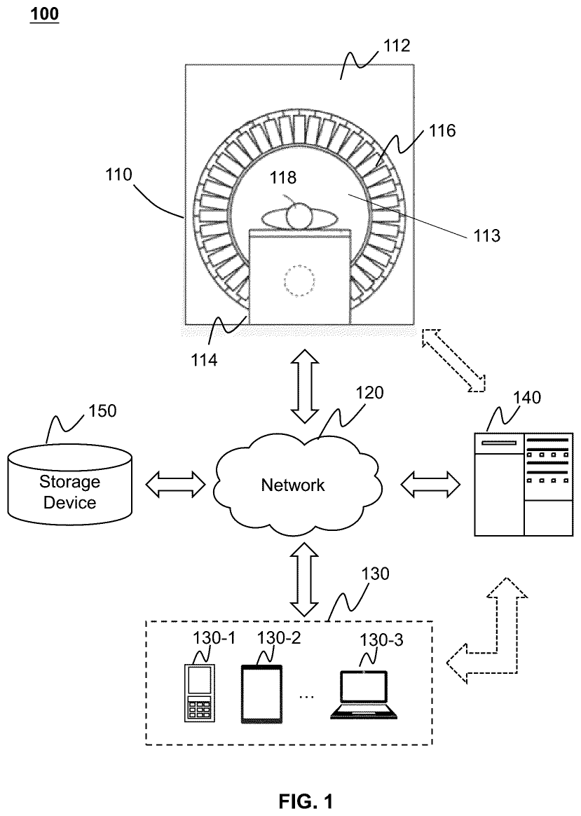

FIG. 1 is a schematic diagram illustrating an exemplary PET system according to some embodiments of the present disclosure. As illustrated in FIG. 1, the PET system 100 may include a PET scanner 110, a network 120, a terminal device 130, a processing device 140, and a storage device 150. The components of the PET system 100 may be connected in various manners.

The PET scanner 110 may be configured to acquire scan data relating to an object. For example, the PET scanner 110 may scan the object or a portion thereof that is located within its detection region and generate the scan data relating to the object or the portion thereof.

In some embodiments, the PET scanner 110 may include a gantry 112, a couch 114, and a detector 116. The gantry 112 may support the detector 116. The couch 114 may be used to support an object 118 to be scanned. The detector 116 may include a plurality of detector rings arranged along an axial direction of the gantry 112. In some embodiments, a detector ring may include a plurality of detector units arranged along the circumference of the detector ring. In some embodiments, the detector 116 may include a scintillation detector (e.g., a cesium iodide detector), a gas detector, or the like, or any combination thereof. In some embodiments, the PET scanner 110 may be a multi-modality scanner, for example, a positron emission tomography-computed tomography (PET-CT) scanner, etc.

The network 120 may facilitate exchange of information and/or data. For example, the processing device 140 may obtain, via the network 120, scan data relating to the object 118 or a portion thereof from the PET scanner 110. In some embodiments, the network 120 may be any type of wired or wireless network, or a combination thereof.

The terminal device 130 may enable interactions between users and components of the PET system 100. The terminal device 130 may include a mobile device 130-1, a tablet computer 130-2, a laptop computer 130-3, or the like, or any combination thereof. In some embodiments, the terminal device 130 may be part of the processing device 140. In some embodiments, the terminal device 130 may be omitted.

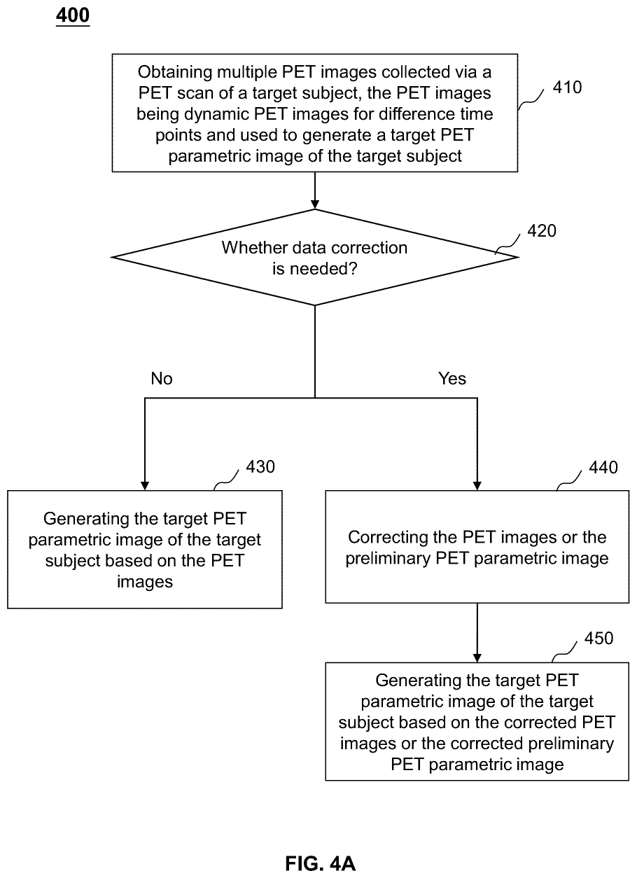

The processing device 140 may process data relating to the PET system 100. In some embodiments, the processing device 140 (e.g., one or more modules illustrated in FIG. 3) may perform the methods of the present disclosure. For example, any one of processes 400-600 and 800-1000 may be implemented as a set of instructions (e.g., an application) stored in a storage device (e.g., the storage device 150). The processing device 140 may execute the set of instructions and may accordingly be directed to perform the processes 400-600 and 800-1000. Merely by way of example, the processing device 140 may obtain scan data (e.g., PET data) of an object (e.g., a human body) from the PET scanner 110 or the storage device 150, and generate a target PET parametric image of the object based on the scan data of the object by performing the process 400.

In some embodiments, the processing device 140 may be a single server or a server group. The server group may be centralized or distributed. In some embodiments, the processing device 140 may be local or remote. Merely for illustration, only one processing device 140 is described in the PET system 100. However, it should be noted that the PET system 100 in the present disclosure may also include multiple processing devices. Thus, operations and/or method steps that are performed by one processing device 140 as described in the present disclosure may also be jointly or separately performed by the multiple processing devices. For example, if in the present disclosure the processing device 140 of the PET system 100 executes both process A and process B, it should be understood that the process A and the process B may also be performed by two or more different processing devices jointly or separately in the PET system 100 (e.g., a first processing device executes process A and a second processing device executes process B, or the first and second processing devices jointly execute processes A and B).

The storage device 150 may store data, instructions, and/or any other information. In some embodiments, the storage device 150 may store data obtained from the processing device 140, the terminal device 130, and/or the PET scanner 110. For example, the storage device 150 may store scan data collected by the PET scanner 110. As another example, the storage device 150 may store the target PET parametric image of the object. In some embodiments, the storage device 150 may store data and/or instructions that the processing device 140 may execute or use to perform exemplary methods described in the present disclosure.

It should be noted that the above description of the PET system 100 is merely provided for the purposes of illustration, and not intended to limit the scope of the present disclosure. For persons having ordinary skills in the art, multiple variations and modifications may be made under the teachings of the present disclosure. For example, the PET system 100 may include one or more additional components and/or one or more components of the PET system 100 described above may be omitted. Additionally or alternatively, two or more components of the PET system 100 may be integrated into a single component. A component of the PET system 100 may be implemented on two or more sub-components.

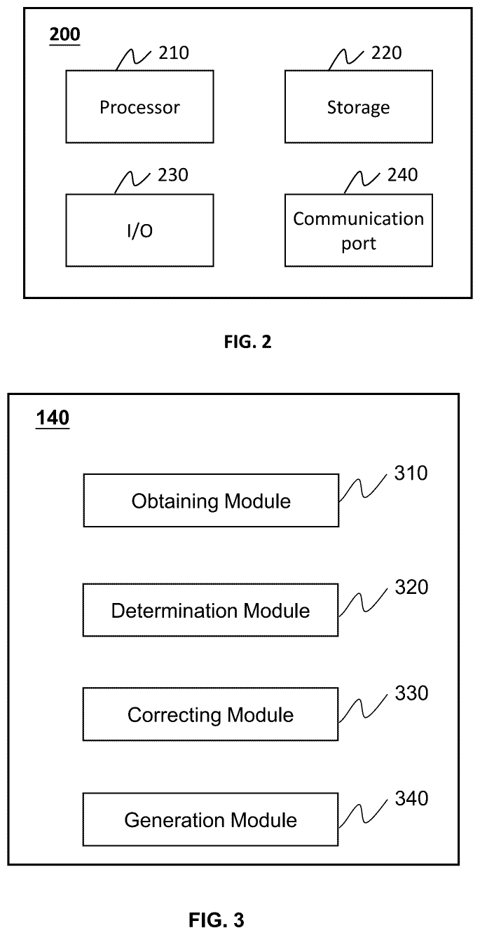

FIG. 2 is a schematic diagram illustrating exemplary hardware and/or software components of an exemplary computing device according to some embodiments of the present disclosure. In some embodiments, the processing device 140 may be implemented on the computing device 200. As illustrated in FIG. 2, the computing device 200 may include a processor 210, a storage 220, an input/output (I/O) 230, and a communication port 240.

The processor 210 may execute computer instructions (program code) and perform functions of the processing device 140 in accordance with techniques described herein. The computer instructions may include routines, programs, objects, components, signals, data structures, procedures, modules, and functions, which perform particular functions described herein. Merely for illustration purposes, only one processor is described in the computing device 200. However, it should be noted that the computing device 200 in the present disclosure may also include multiple processors, and thus operations of a method that are performed by one processor as described in the present disclosure may also be jointly or separately performed by the multiple processors.

The storage 220 may store data/information obtained from the PET scanner 110, the terminal device 130, the storage device 150, or any other component of the PET system 100. In some embodiments, the storage 220 may include a mass storage device, a removable storage device, a volatile read-and-write memory, a read-only memory (ROM), or the like, or any combination thereof. In some embodiments, the storage 220 may store one or more programs and/or instructions to perform exemplary methods described in the present disclosure.

The I/O 230 may input or output signals, data, or information. In some embodiments, the I/O 230 may enable user interaction with the processing device 140. In some embodiments, the I/O 230 may include an input device and an output device.

The communication port 240 may be connected to a network (e.g., the network 120) to facilitate data communications. The communication port 240 may establish connections between the processing device 140 and the PET scanner 110, the terminal device 130, or the storage device 150. The connection may be a wired connection, a wireless connection, or combination of both that enables data transmission and reception.

It should be noted that the above description of the computing device 200 is merely provided for the purposes of illustration, and not intended to limit the scope of the present disclosure. For persons having ordinary skills in the art, multiple variations and modifications may be made under the teachings of the present disclosure.

FIG. 3 is a block diagram illustrating exemplary processing device 140 according to some embodiments of the present disclosure.

As shown in FIG. 3, the processing device 140 may include an obtaining module 310, a determination module 320, a correcting module 330, and a generation module 340. As described in FIG. 1, the PET system 100 in the present disclosure may also include multiple processing devices, and the obtaining module 310, the determination module 320, the correcting module 330, and the generation module 340 may be components of different processing devices.

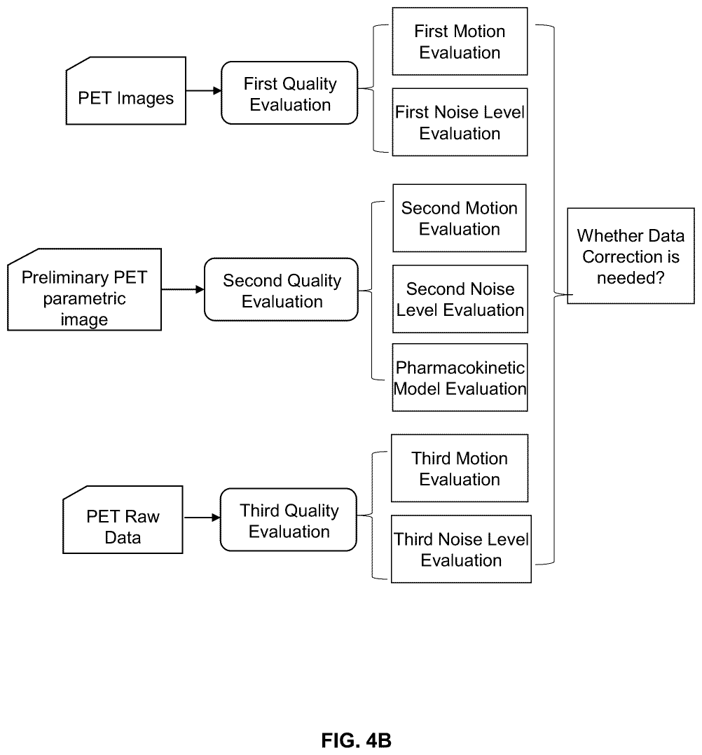

The obtaining module 310 may be configured to obtain information relating to the PET system 100. For example, the obtaining module 310 may obtain multiple PET images collected via a PET scan of a target subject. The PET images may be dynamic PET images for difference time periods during the PET scan and used to generate a target PET parametric image of the target subject. More descriptions regarding the obtaining of the multiple PET images may be found elsewhere in the present disclosure. See, e.g., operation 410 in FIG. 4, and relevant descriptions thereof.

The determination module 320 may be configured to determine whether data correction needs to be performed in a process of generating the target PET parametric image (referred to as whether data correction is needed for brevity). More descriptions regarding the determining whether data correction is needed may be found elsewhere in the present disclosure. See, e.g., operation 420 in FIG. 4, and relevant descriptions thereof.

The correcting module 330 may be configured to correct the PET images or a preliminary PET parametric image. More descriptions regarding the correction of the PET images or the preliminary PET parametric image may be found elsewhere in the present disclosure. See, e.g., operation 440 in FIG. 4, and relevant descriptions thereof.

The generation module 340 may be configured to generate the target PET parametric image of the target subject. For example, the generation module 340 may generate the target PET parametric image of the target subject based on the PET images. As another example, the generation module 340 may generate the target PET parametric image of the target subject based on the corrected PET images or the corrected preliminary PET parametric image. More descriptions regarding the generation of the target PET parametric image of the target subject may be found elsewhere in the present disclosure. See, e.g., operations 430 and 450 in FIG. 4, and relevant descriptions thereof.

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. In some embodiments, any one of the modules may be omitted or divided into two or more units. For instance, the obtaining module 310 may be divided into two units configured to acquire different data. In some embodiments, the processing device 140 may include one or more additional modules, such as a storage module (not shown) for storing data.

FIG. 4A is a flowchart illustrating an exemplary process 400 for parametric imaging in PET according to some embodiments of the present disclosure.

In 410, the processing device 140 (e.g., the obtaining module 310) may obtain multiple PET images collected via a PET scan of a target subject, the PET images being dynamic PET images for difference time periods during the PET scan and used to generate a target PET parametric image of the target subject.

As used herein, a PET parametric image may reflect a kinetic parameter of a tracer in a subject. As used herein, the term “kinetic parameter” refers to a physiological parameter associated with the kinetics of a tracer after the tracer is injected into a subject. For instance, the kinetic parameter may include a transportation rate of the tracer from a plasma to tissue (or referred to as a K₁ parameter of the tracer), a transportation rate of the tracer from the tissue to the plasma (or referred to as a k₂ parameter of the tracer), a concentration of plasma in the tissue, a perfusion rate of the tracer, a receptor binding potential of the tracer, a Ki parameter of the tracer, or the like, or any combination thereof. The parametric image may aid the evaluation of the physiology (functionality) and/or anatomy (structure) of an organ and/or tissue in the subject.

In some embodiments, the parametric image may present a value of a kinetic parameter corresponding to one or more time points (or time periods) during the PET scan. For example, the parametric image may include one or more static images corresponding to one or more time points.

The target PET parametric image refers to a PET parametric image of the target subject to be generated whose quality satisfies requirements. For example, the noise level of the target PET parametric image needs to be below a noise level threshold. As another example, the target PET parametric image should have little or no artifacts (e.g., motion artifacts).

The target subject may include a patient, an animal, a phantom, or a portion thereof including, for example, an artificial limb, an artificial heart, a tumor, any structure or organ that may be examined.

The multiple PET images may reflect the activity change of a tracer used in the PET scan with respect to time. In some embodiments, the processing device 140 may obtain multiple sets of PET raw data of the target subject from a PET scanner or a storage device (e.g., the storage device 150 or an external source). Each set of PET raw data may be collected in one time period during the PET scan. Further, for each set of PET raw data, the processing device 140 may reconstruct one PET image of the multiple PET images based on the set of PET raw data. The PET raw data may be collected by scanning a region of interest (ROI) of the target subject via the PET scanner 110. In some embodiments, the sets of PET raw data may include information relating to radiation events during the PET scan such as a response of line (LOR), coincidence event counts, etc. The ROI may include any part of the target subject. For example, the ROI may include a whole body of the target subject. Alternatively, the ROI may be a portion of the target subject, such as a brain, a lung, a liver, a kidney, a bone, etc.

In some embodiments, the processing device 140 may directly generate the multiple PET images by reconstructing the sets of PET raw data using a same reconstruction algorithm. Exemplary reconstruction algorithms may include a maximum-likelihood reconstruction of attenuation and activity (MLAA) algorithm, an iterative reconstruction algorithm (e.g., a statistical reconstruction algorithm), a Fourier slice theorem algorithm, a filtered back projection (FBP) algorithm, a compressed sensing (CS) algorithm, a fan-beam reconstruction algorithm, a maximum likelihood expectation maximization (MLEM) algorithm, an ordered subset expectation maximization (OSEM) algorithm, a maximum a posterior (MAP) algorithm, an analytic reconstruction algorithm, or the like, or any combination thereof.

In some embodiments, for each set of PET raw data, the processing device 140 may determine a coincidence event count based on the set of PET raw data, and determine a reconstruction algorithm for reconstructing the set of PET raw data based on the coincidence event count. Further, the processing device 140 may generate the PET images by reconstructing the sets of PET raw data using their respective reconstruction algorithms.

For example, for each set of PET raw data, the processing device 140 may obtain the coincidence event count corresponding to the set of PET raw data from the set of PET raw data. The coincidence event count of a set of PET raw data may reflect the noise level of the set of PET raw data. Specifically, the greater the coincidence event count corresponding to a set of PET raw data is, the lower the noise level the set of PET raw data may be. If the coincidence event count corresponding to a set of PET raw data is smaller than a count threshold, the processing device 140 may determine a reconstruction algorithm with noise reduction function as the reconstruction algorithm for reconstructing the set of PET raw data. Exemplary reconstruction algorithms with noise reduction function may include a reconstruction algorithm incorporated with a regularization term, a Deep Progressive Reconstruction (DPR) algorithm, or the like. In this way, the noise level of the PET images can be reduced, so subsequent noise evaluation (e.g., the first noise evaluation or the third noise evaluation) is not required, thereby improving the efficiency of parametric imaging.

In 420, the processing device 140 (e.g., the determination module 320) may determine whether data correction needs to be performed in a process of generating the target PET parametric image (referred to as whether data correction is needed for brevity).

In some embodiments, the processing device 140 may determine whether data correction is needed based on one or more of the PET images, a preliminary PET parametric image, or the sets of PET raw data. In some embodiments, determining whether data correction is needed may include one or more of determining whether motion correction is needed, determining whether noise correction is needed, or determining whether pharmacokinetic model correction is needed.

Specifically, the processing device 140 may perform one or more of a first quality evaluation on the PET images, a second quality evaluation on the preliminary PET parametric image, or a third quality evaluation on the sets of PET raw data. Further, the processing device 140 may determine whether data correction is needed based on the result(s) of one or more of the first quality evaluation, the second quality evaluation, or the third quality evaluation. In some embodiments, the first quality evaluation may include a first motion evaluation, a first noise level evaluation, or the like, or any combination thereof. The second quality evaluation may include a second motion evaluation, a second noise level evaluation, and a pharmacokinetic model evaluation, or the like, or any combination thereof. The third quality evaluation may include a third motion evaluation, a third noise level evaluation, or the like, or any combination thereof. More descriptions regarding the determination of whether data correction is needed may be found elsewhere in the present disclosure. See, e.g., FIG. 4B and relevant descriptions thereof. In this way, whether data correction is needed may be determined based on quality evaluations of multiple dimensions, which may obtain an accurate evaluation result, thereby ensuring the quality of the target PET parametric image.

The preliminary PET parametric image refers to a preliminarily determined PET parametric image generated by processing the corrected PET images described in operations 440 and 450 or the PET images using at least one pharmacokinetic model. For example, the corrected PET images may be generated by performing the motion correction and/or the noise reduction operation on the PET images. The pharmacokinetic model may indicate a metabolism situation of the tracer injected into the target subject. Pharmacokinetics may be used to quantitatively study an absorption, a distribution, a metabolism, and/or an excretion of a drug in an object, and describe the tracer concentration changes over time using mathematical principles or methods. The pharmacokinetic model may be used to quantitatively study kinetic processes of the tracer in the object. In some embodiments, the pharmacokinetic model may include a LOGAN model, 2TIC model, a 2TC model, or the like.

In some embodiments, for each physical point of the target subject, the processing device 140 may determine a pharmacokinetic model corresponding to the physical point, and further determine a PET parametric value of the physical point based on pixel values corresponding to the physical point in the corrected PET images or the PET images using the pharmacokinetic model of the physical point. For example, the processing device 140 may input the pixel values corresponding to the physical point in the PET images into the pharmacokinetic model of the physical point, and the pharmacokinetic model of the physical point may output the PET parametric value of the physical point. Then, the processing device 140 may generate the preliminary PET parametric image based on the PET parametric value of each physical point of the target subject.

In some embodiments, the pharmacokinetic models corresponding to all physical points of the target subject may be the same. In such cases, the pharmacokinetic model evaluation may be performed.

In some embodiments, the pharmacokinetic models corresponding to different physical points may be different. In this case, the pharmacokinetic model evaluation is not required, thereby improving the efficiency of parametric imaging.

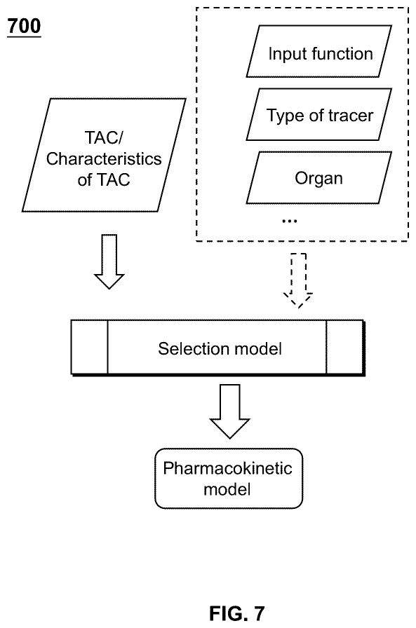

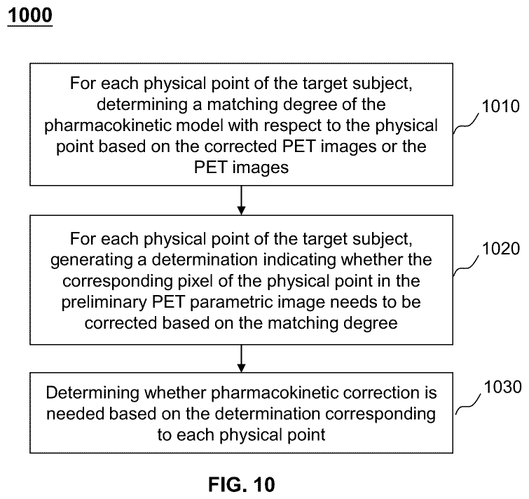

Specifically, for each physical point of the target subject, the processing device 140 may determine a TAC of the physical point based on pixel values corresponding to the physical point in the PET images. In some embodiments, the TAC of the physical point may be a fitting TAC or a measurement TAC. More descriptions regarding the determination of the TAC of the physical point may be found elsewhere in the present disclosure. See, operation 710 in FIG. 7, operation 1010 in FIG. 10, and the descriptions thereof. Further, the processing device 140 may select the pharmacokinetic model corresponding to the physical point based on the TAC of the physical point from multiple candidate pharmacokinetic models.

In some embodiments, the processing device 140 may determine characteristics of the TAC of the physical point. Exemplary characteristics may include a peak value, a peak width, a time required to reach the peak value, or the like, or any combination thereof. The processing device 140 may obtain a corresponding relationship between characteristics of TAC and pharmacokinetic models from a storage device (e.g., the storage device 150 or an external source). Further, the processing device 140 may determine the pharmacokinetic model corresponding to the physical point according to the corresponding relationship and the characteristics of the TAC of the physical point.

In some embodiments, the pharmacokinetic model corresponding to the physical point may be determined by processing the TAC of the physical point using a selection model. The selection model may be a trained model (e.g., a machine leaning model) for selecting a suitable pharmacokinetic model for a physical point. Specifically, the processing device 140 may input a model input into the selection model, and the selection model may output the pharmacokinetic model corresponding to the physical point. The model input at least includes the TAC of the physical point and/or the characteristics of the TAC of the physical point. In some embodiments, the model input may further include other information. For example, the model input may further include an input function of the physical point, a type of tracer used in the PET scan, an organ corresponding to the physical point, or the like, or any combination thereof. The input function may reflect a concentration change of the tracer in the target subject during the PET scan. In some embodiment, the input function may be an image-derived input function or a population-based input function. As used herein, an image-derived input function refers to an input function that is determined based on one or more PET images of a subject. A population-based input function refers to an input function of a subject that is determined based on a plurality of sample input functions corresponding to a plurality of sample subjects other than the subject.

Merly by way of example, FIG. 7 is a schematic diagram illustrating an exemplary process 700 for determining a pharmacokinetic model corresponding to a physical point using the selection mode according to some embodiments of the present disclosure. As shown in FIG. 7, the TAC of the physical point and/or the characteristics of the TAC of the physical point may be input into the selection model. In some embodiments, one or more of the input function of the physical point, the type of tracer used in the PET scan, or the organ corresponding to the physical point may be also input into the selection model together with the TAC or the characteristics of the TAC. The selection model may output the pharmacokinetic model corresponding to the physical point.

In some embodiments, the processing device 140 may obtain the selection model from one or more components of the PET system 100 (e.g., the storage device 150, the terminals(s) 130) or an external source via a network (e.g., the network 120). For example, the selection model may be previously trained by a computing device (e.g., the processing device 140), and stored in a storage device (e.g., the storage device 150, the storage 220) of the PET system 100. The processing device 140 may access the storage device and retrieve the selection model.

In some embodiments, the selection model may be generated by training a preliminary model using training samples. Each training sample may correspond to a sample physical point. In some embodiments, a training sample may be determined by performing the following operations. A sample TAC of the sample physical point may be determined based on sample PET images collected in a sample PET scan of a sample subject. In some embodiments, the determination of the sample TAC of the sample physical point may be performed in a similar manner as that of the TAC of the physical point described in operation 710 in FIG. 7, and the descriptions thereof are not repeated here. Further, predicted TACs of the sample physical point corresponding to the candidate pharmacokinetic models may be determined by processing the sample PET images using the candidate pharmacokinetic models. In some embodiments, the determination of the predicted TACs may be performed in a similar manner as that of the second TAC of the physical point described in operation 1010 in FIG. 10, and the descriptions thereof are not repeated here. Then, a recommended pharmacokinetic model may be determined from the candidate pharmacokinetic models based on the sample TAC and the predicted TACs. Specifically, a similarity between the sample TAC and each predicted TAC may be determined. A candidate pharmacokinetic model corresponding to the predicted TAC with the largest similarity may be designated as the recommended pharmacokinetic model. The sample TAC may be used to determine a training input of the training sample and the recommended pharmacokinetic model may be designated as a training label of the training sample. The training input may be similar to the input of the selection input during application.

In some embodiments, in response to determining that data correction is needed, the processing device 140 may perform operations 440 and 450; in response to determining that data correction is not needed, the processing device 140 may perform operation 430.

In 430, the processing device 140 (e.g., the generation module 340) may generate the target PET parametric image of the target subject based on the PET images.

For example, the processing device 140 may designate the preliminary PET parametric image as the target PET parametric image.

In 440, the processing device 140 (e.g., the correcting module 330) may correct the PET images or the preliminary PET parametric image.

In some embodiments, if the result of the motion evaluation (e.g., the first motion evaluation, the second motion evaluation, or the third motion evaluation) indicates that the target subject has an obvious movement or deformation during the PET scan, which indicates that there is a high probability of motion artifacts in the PET images or the preliminary PET parametric image, the processing device 140 may perform a motion correction on the PET images.

For example, the processing device 140 may use a motion artifact correction algorithm to correct motion artifacts in the PET images. As another example, the processing device 140 may determine a reference image. The reference image may be one of the PET images or a computed tomography (CT) image of the target subject acquired by a CT scan of the target subject. The CT scan may be performed before or after the PET scan with the target subject keeps essentially the same patient position. The fields of view (FOVs) of the CT scan and the PET scan may at least cover the same ROI of the target subject. Further, the processing device 140 may register the reference image with each PET image based on a registration algorithm. In some embodiments, the registration may be performed based on a rigid transformation algorithm, a non-rigid transformation algorithm, an affine transformation algorithm, a projection transformation algorithm, a nonlinear transformation algorithm, an optical-flow-based registration, a similarity measurement, or the like, or any combination thereof. For example, if the ROI of the target subject is the head, the registration may be performed based on the rigid transformation algorithm. As another example, if the ROI of the target subject is the liver or lungs, the registration may be performed based on the non-rigid transformation algorithm.

In some embodiments, if the second motion evaluation indicates that the target subject has an obvious movement or deformation during the PET scan, the processing device 140 may perform a motion correction on the preliminary PET parametric image. For example, the processing device 140 may register the reference image with preliminary PET parametric image based on the registration algorithm or use a motion artifact correction algorithm to correct motion artifacts in the preliminary PET parametric image.

In some embodiments, if the result of the first noise level evaluation indicates that the noise level of the PET images is relatively large, the processing device 140 may perform a noise reduction operation on the PET images. In some embodiments, if the result of the second noise level evaluation indicates that the noise level of the preliminary PET parametric image is relatively large, the processing device 140 may perform a noise reduction operation on the preliminary PET parametric image. In some embodiments, if the result of the third noise level evaluation indicates that the noise level of the sets of PET raw data is relatively large, the processing device 140 may perform the noise reduction operation on the sets of PET raw data. Then, the processing device 140 may generate the corrected PET images by reconstructing the denoised PET raw data using the reconstruction algorithm described in operation 410. Alternatively, the processing device 140 may perform a noise reduction operation on the PET images.

In some embodiments, if the result of the pharmacokinetic model evaluation indicates that the pharmacokinetic model for generating the preliminary PET parametric image does not satisfy requirements, the processing device 140 may perform a parametric correction on the preliminary PET parametric image. For each target physical point that need to be corrected, the processing device may determine a new pharmacokinetic model corresponding to the target physical point in a similar manner as that described in operation 420 in FIG. 4. Further, the processing device 140 may determine a new PET parametric value of the target physical point based on pixel values corresponding to the target physical point in the PET images using the new pharmacokinetic model of the target physical point. Then, the processing device 140 may update the parametric values of the target physical points with the new PET parametric values of the physical points.

In some embodiments, if at least two of the motion correction, the noise reduction operation, or the parametric correction need to be performed, they can be performed in any order. For example, after the motion correction and/or the noise reduction operation are performed on the PET images, the preliminary PET parametric image is generated based on the corrected PET images. If the result of the pharmacokinetic model evaluation indicates that the pharmacokinetic model for generating the preliminary PET parametric image does not satisfy requirements, the processing device 140 may perform further the parametric correction on the preliminary PET parametric image.

In 450, the processing device 140 (e.g., the generation module 340) may generate the target PET parametric image of the target subject based on the corrected PET images or the corrected preliminary PET parametric image.

In some embodiments, the processing device 140 may generate the target PET parametric image of the target subject based on the corrected PET images in a similar manner as how the preliminary PET parametric image is generated based on the PET images as described in operation 420. In some embodiments, the processing device 140 may designate the corrected preliminary PET parametric image as the target PET parametric image of the target subject.

As described elsewhere in the present disclosure, the conventional parametric imaging techniques usually have low imaging efficiency and high imaging cost. Compared with the conventional parametric imaging techniques, the methods and systems of the present disclosure may determine whether data correction needs to be performed in advance, automatically correct the PET images or the preliminary PET parametric image, and generate the target PET parametric image based on the corrected PET images or the corrected preliminary PET parametric image. In this way, an accurate target PET parametric image can be obtained, and the efficiency of the parametric imaging can be improved since no new PET imaging is required.

In some embodiments, according to the result of the motion evaluation (e.g., the first motion evaluation, the second motion evaluation, or the third motion evaluation), the motion correction may be performed on the PET images or the preliminary PET parametric image, thereby reducing or eliminating motion artifacts in the target PET parametric image.

In some embodiments, according to the result of the first noise level evaluation, the second noise level evaluation, or the third noise level evaluation, the noise reduction operation may be performed on the PET images or the sets of PET raw data, thereby reducing or eliminating the noise in the target PET parametric image.

In some embodiments, according to the result of the pharmacokinetic model evaluation, the parametric correction may be performed on the preliminary PET parametric image, thereby improving the accuracy of the target PET parametric image.

FIG. 4B is a schematic diagram illustrating an exemplary process for determine whether data correction is needed according to some embodiments of the present disclosure.

As shown in FIG. 4B, the processing device 140 may perform one or more of a first quality evaluation on the PET images, a second quality evaluation on the preliminary PET parametric image, or a third quality evaluation on the sets of PET raw data. Further, the processing device 140 may determine whether data correction is needed based on the result(s) of one or more of the first quality evaluation, the second quality evaluation, or the third quality evaluation.

In some embodiments, the first quality evaluation may include a first motion evaluation, a first noise level evaluation, or the like, or any combination thereof. The second quality evaluation may include a second motion evaluation, a second noise level evaluation, and a pharmacokinetic model evaluation, or the like, or any combination thereof. The third quality evaluation may include a third motion evaluation, a third noise level evaluation, or the like, or any combination thereof.

In some embodiments, a motion evaluation (e.g., the first motion evaluation, the second motion evaluation, or the third motion evaluation) may be used to evaluate the movement or deformation of the target subject during the PET scan. If a result of the motion evaluation indicates that the target subject has an obvious movement or deformation during the PET scan, the processing device 140 may determine that motion correction is needed; if the motion evaluation result indicates that the target subject has no obvious movement or deformation during the PET scan, the processing device 140 may determine that motion correction is not needed.

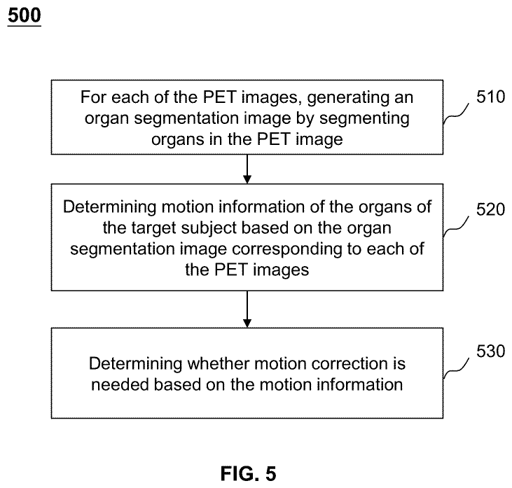

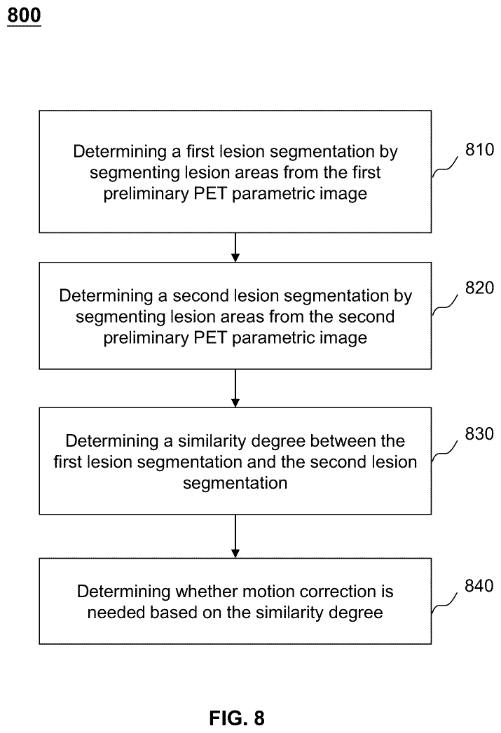

In some embodiments, the first motion evaluation may be performed by performing operations 510 and 520 in FIG. 5. In some embodiments, the second motion evaluation may be performed by operations 810-830 in FIG. 8.

In some embodiments, the third motion evaluation may be performed by the following way. For each set of PET raw data, the processing device 140 may generate a histoimage corresponding to the set of PET raw data. Further, the processing device 140 may determine motion information of organs of the target subject based on the histoimage corresponding to each set of PET raw data. For example, the motion information may be determined by registering the histoimages. As another example, for each histoimage, the processing device 140 may generate an organ segmentation image by segmenting organs in the histoimage. Further, the processing device 140 may determine the motion information of the organs of the target subject based on the organ segmentation image corresponding to each histoimage. The determination of the motion information based on the organ segmentation image corresponding to each histoimage may be performed in a similar manner as that described in operation 520, and the descriptions thereof are not repeated here.

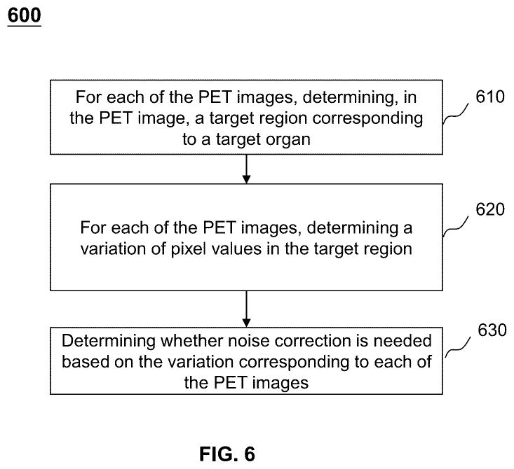

In some embodiments, the first noise level evaluation may be used to evaluate a noise level of the PET images. If a result of the first noise level evaluation indicates that the noise level of the PET images is relatively large, the processing device 140 may determine that noise correction is needed; if the result of the first noise level evaluation indicates that the noise level of the PET images is relatively small, the processing device 140 may determine that noise correction is not needed. In some embodiments, the first noise level evaluation may be performed by operations 610 and 620 in FIG. 6.

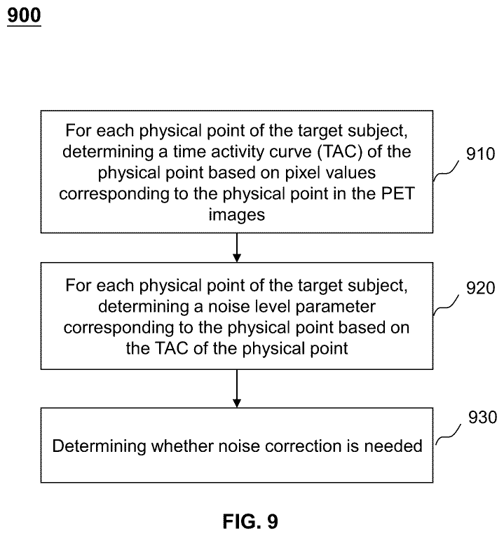

In some embodiments, the second noise level evaluation may be used to evaluate a noise level of the preliminary PET parametric image. If a result of the second noise level evaluation indicates that the noise level of the preliminary PET parametric image is relatively large, the processing device 140 may determine that noise correction is needed; if the result of the second noise level evaluation indicates that the noise level of the preliminary PET parametric image is relatively small, the processing device 140 may determine that noise correction is not needed. In some embodiments, the second noise level evaluation may be performed by operations 910-930 in FIG. 9.

In some embodiments, the pharmacokinetic model evaluation may be used to evaluate whether the pharmacokinetic model for generating the preliminary PET parametric image satisfies requirements. If a result of the pharmacokinetic model evaluation indicates that the pharmacokinetic model for generating the preliminary PET parametric image does not satisfy requirements, the processing device 140 may determine that pharmacokinetic model correction is needed; if the result of the pharmacokinetic model evaluation indicates that the pharmacokinetic model for generating the preliminary PET parametric image satisfies requirements, the processing device 140 may determine that pharmacokinetic model correction is not needed.

In some embodiments, the pharmacokinetic model evaluation may be performed by performing operations 1010 and 1020 in FIG. 10.

In some embodiments, the third noise level evaluation may be used to evaluate the noise level of the of the sets of PET raw data. If a result of the third noise level evaluation indicates that the noise level of the sets of PET raw data is relatively large, the processing device 140 may determine that noise correction is needed; if the result of the third noise level evaluation indicates that the noise level of the sets of PET raw data is relatively small, the processing device 140 may determine that noise correction is not needed.

In some embodiments, the third noise level evaluation may be performed by the following way. For each set of PET raw data, the processing device 140 may determine a coincidence event count based on the set of PET raw data, and further determine a noise level parameter of the set of PET raw data based on the coincidence event count corresponding to the set of PET raw data. Specifically, as described in operation 410, the sets of PET raw data may include information relating to radiation events during the PET scan such as a response of line (LOR), coincidence event counts, etc. For each set of PET raw data, the processing device 140 may obtain the coincidence event count corresponding to the set of PET raw data from the set of PET raw data. The greater the coincidence event count corresponding to a set of PET raw data is, the smaller the noise level parameter of the set of PET raw data may be. For each set of PET raw data, the processing device 140 may determine the noise level parameter of the set of PET raw data according to the coincidence event count corresponding to the set of PET raw data.

Then, the processing device 140 may determine whether noise correction is needed based on the noise level parameter of each set of PET raw data. Specifically, the processing device 140 may determine a total noise level parameter of the sets of PET raw data based on the noise level parameter of each set of PET raw data. For example, the processing device 140 may designate an average (or a median, or a mode) of the noise level parameters of the sets of PET raw data as the total noise level parameter of the sets of PET raw data. If the total noise level parameter is greater than a noise level parameter threshold, the processing device 140 may determine that the noise level of the of the sets of PET raw data is relatively large, and noise correction is needed. If the total noise level parameter is not greater than the noise level parameter threshold, the processing device 140 may determine that the noise level of the of the sets of PET raw data is relatively small, and noise correction is not needed.

In some embodiments, for a same type of evaluation (e.g., the motion evaluation, the noise evaluation), the processing device 140 may determine whether the corresponding correction is needed based on the at least two of the first quality evaluation, the second quality evaluation, or the third quality evaluation. For example, the processing device 140 may perform the first quality evaluation on the PET images and the second quality evaluation on the preliminary PET parametric image. Further, the processing device 140 may determine whether the data correction is needed based on results of the first quality evaluation and the second quality evaluation. In some embodiments, if anyone of the first quality evaluation and the second quality evaluation indicates that data correction is needed, the processing device 140 may determine that data correction is needed.

In some embodiments, the result of the first quality evaluation includes one or more first quality evaluation parameters, the result of the second quality evaluation includes one or more second quality evaluation parameters. The one or more first quality evaluation parameters may include a motion parameter (e.g., the motion information described in operation 520), a noise level parameter (e.g., the variation of pixel values described in operation 620, the noise level parameter described in operation 720), or the like. The one or more second quality evaluation parameters may include a motion parameter (e.g., the similarity described in operation 930), a pharmacokinetic model parameter (e.g., the determination result indicating whether the corresponding pixel of the physical point needs to be corrected described in operation 1020), or the like.

If the first quality evaluation and the second quality evaluation have contradictory results regarding the same type of evaluation (e.g., the motion evaluation or the noise evaluation), the processing device 140 may determine first difference information between the one or more first evaluation parameters and their corresponding threshold values, second difference information between the one or more second evaluation parameters and their corresponding threshold values. Further, the processing device 140 may determine whether data correction is needed based on the first difference information and the second difference information. For example, for the motion evaluation, if the result of the first motion evaluation indicates that the total displacements of one or more organs are greater than the second displacement threshold and the result of the second motion evaluation of indicates that the similarity is smaller than the similarity threshold, the processing device 140 may determine first differences between the total displacements and the second displacement threshold and a second difference between the similarity and the similarity threshold. The processing device 140 may determine whether motion correction is needed based on the result corresponding to the maximum difference among the first differences and the second difference.

FIG. 5 is a flowchart illustrating an exemplary process 500 for determining whether motion correction is needed according to some embodiments of the present disclosure. In some embodiments, one or more operations of the process 500 may be performed to achieve at least part of operation 420 as described in connection with FIG. 4.

In 510, for each of the PET images, the processing device 140 (e.g., the determination module 320) may generate an organ segmentation image by segmenting organs in the PET image.

In some embodiments, the organ segmentation image may be represented as a segmentation mask of the organs generated based on the PET image. For example, portions corresponding to the organs may be identified in the PET image, and the segmentation mask may be generated based on the identified portion.

In some embodiments, the organs may be segmented from the PET image manually by a user (e.g., a doctor, an imaging specialist, a technician) by, for example, drawing a bounding box on the PET image displayed on a user interface. Alternatively, the PET image may be segmented by the processing device 140 automatically according to an image analysis algorithm (e.g., an image segmentation algorithm). For example, the processing device 140 may perform image segmentation on the PET image using an image segmentation algorithm (e.g., a machine learning-based segmentation algorithm). Alternatively, the organs may be segmented by the processing device 140 semi-automatically based on an image analysis algorithm in combination with information provided by a user. Exemplary information provided by the user may include a parameter relating to the image analysis algorithm, a position parameter relating to a region to be segmented, an adjustment to, or rejection or confirmation of a preliminary segmentation result generated by the processing device 140, etc.

In 520, the processing device 140 (e.g., the determination module 320) may determine motion information of the organs of the target subject based on the organ segmentation image corresponding to each of the PET images.

In some embodiments, the motion information of the organs may include displacements of each organ corresponding to any two adjacent PET images, a total displacement of the organ, motion fields corresponding to any two adjacent PET images, or the like, or any combination thereof.

In some embodiments, for each organ, the processing device 140 determine the displacement of the organ corresponding to any two adjacent PET images according to the organ segmentation images corresponding to any two adjacent PET images. In some embodiments, for each organ, the processing device 140 determine a sum of the displacements of the organ corresponding to any two adjacent PET images as the total displacement of the organ.

In some embodiments, for any two adjacent PET images, the processing device 140 may determine a motion field corresponding to the two adjacent PET images by registering the two adjacent PET images using a registration algorithm. In some embodiments, for each organ, the processing device 140 determine a sum of the motion fields of the organ corresponding to any two adjacent PET images as the total motion field of the organ.

In 530, the processing device 140 (e.g., the determination module 320) may determine whether motion correction is need based on the motion information of the organs.

In some embodiments, if there are one or more organs with displacements or motion fields greater than a first displacement threshold, the processing device 140 may determine that the target subject has an obvious movement or deformation during the PET scan, and motion correction is needed for the PET images; if there is no organ with a displacement or motion field greater than the first displacement threshold; the processing device 140 may determine the target subject has no obvious movement or deformation during the PET scan, and motion correction is not needed for the PET images. In some embodiments, if there are one or more organs whose total displacements or total motion fields are greater than a second displacement threshold, the processing device 140 may determine that the target subject has an obvious movement or deformation during the PET scan, and motion correction is needed for the PET images; if there is no organ whose total displacement or total motion field is greater than the second displacement threshold, the processing device 140 may determine the target subject has no obvious movement or deformation during the PET scan, and motion correction is not needed for the PET images.

FIG. 6 is a flowchart illustrating an exemplary process 600 for determining whether noise correction is needed according to some embodiments of the present disclosure. In some embodiments, one or more operations of the process 600 may be performed to achieve at least part of operation 420 as described in connection with FIG. 4.

In 610, for each of the PET images, the processing device 140 (e.g., the determination module 320) may determine, in the PET image, a target region corresponding to a target organ.

In some embodiments, the target organ may be an organ with a relatively large number of pixels and a relatively uniform activity value, which can reflect the overall noise level of the PET image. For example, the target organ may the liver of the target subject. In some embodiments, the processing device 140 may determine the target region by segmenting the target organ from the PET image.

In 620, for each of the PET images, the processing device 140 (e.g., the determination module 320) may determine a variation of pixel values in the target region.

In some embodiments, the variation of pixel values may be represented via a coefficient of variation (COV) of the pixel values. As used herein, a COV of pixel values refers to a ratio of a standard deviation of the pixel values to an average of the pixel values. The processing device 140 may determine the COV of pixel values in the target region, and designate the COV of pixel values as the variation of pixel values in the target region. It should be noted that under ideal conditions, the medium in an organ is evenly distributed, the variation of the pixel values in the organ is small, and the corresponding COV of the pixel values in the organ is small, while the noise in the image increases the COV and the variation of the pixel values in the organ. Therefore, the COV corresponding to a PET image may indicate the noise level in the PET image. The higher the COV, the higher the noise level in the PET image.

The processing device 140 may designate the variation of pixel values in the target region as the variation corresponding to the PET image. In this way, the variation corresponding to the PET image can be obtained by analyzing the COV of the local region (i.e., the target region) of the PET image, which can reduce the amount of data processing and save computing resources.

In 630, the processing device 140 (e.g., the determination module 320) may determine whether noise correction is needed based on the variation corresponding to each of the PET images.

In some embodiments, the processing device 140 may determine an average of the variations corresponding to the PET images, and designate the average as the total variation corresponding to the PET images. In response to determining that the total variation is greater than a variation threshold, the processing device 140 may determine that the noise level of the PET images is relatively large, and noise correction is needed for the PET images. In response to determining that the total variation is not greater than the variation threshold, the processing device 140 may determine that the noise level of the PET images is relatively small, and noise correction is not needed for the PET images.

FIG. 8 is a flowchart illustrating an exemplary process 800 for determining whether motion correction is needed according to some embodiments of the present disclosure. In some embodiments, one or more operations of the process 800 may be performed to achieve at least part of operation 420 as described in connection with FIG. 4.

In some embodiments, the preliminary PET parametric image may include a first preliminary PET parametric image and a second preliminary PET parametric image corresponding to different PET parameters. For example, the first preliminary PET parametric image and the second preliminary PET parametric image may be a Ki parametric image and an intercept parametric image, respectively.

In 810, the processing device 140 (e.g., the determination module 320) may determine a first lesion segmentation result by segmenting lesion areas from the first preliminary PET parametric image.

In 820, the processing device 140 (e.g., the determination module 320) may determine a second lesion segmentation result by segmenting lesion areas from the second preliminary PET parametric image.

In some embodiments, a lesion segmentation result may be represented as a segmentation mask of the lesion areas generated based on the corresponding preliminary PET parametric image.

In some embodiments, the determination of the first and second lesion segmentation results may be performed in a similar manner as that of the organ segmentation image described in operation 510, and the descriptions thereof are not repeated here. For example, the lesion areas may be segmented from the first preliminary PET parametric image manually by a user (e.g., a doctor, an imaging specialist, a technician) or automatically by the processing device 140 according to an image segmentation algorithm (e.g., a lesion segmentation or detection algorithm).

In 830, the processing device 140 (e.g., the determination module 320) may determine a similarity between the first lesion segmentation result and the second lesion segmentation result.

The similarity between the first lesion segmentation result and the second lesion segmentation result may be represented by such as mutual information (MI) similarity, normalized mutual information (NMI) similarity, a mean squared error (MSE), a structural similarity (SSIM), a Dice similarity coefficient (DSC), etc., between the first lesion segmentation result and the second lesion segmentation result. In some embodiments, the processing device 140 may determine the similarity between the first lesion segmentation result and the second lesion segmentation result using a similarity algorithm. Exemplary similarity algorithms may include an algorithm based on histoimage, an algorithm based on Euclidean distance, an algorithm based on Pearson correlation coefficient, an algorithm based on cosine similarity, a hash algorithm, or the like.

In 840, the processing device 140 (e.g., the determination module 320) may determine whether motion correction is needed based on the similarity.

In some embodiments, if the similarity is smaller than a similarity threshold, the processing device 140 may determine that the target subject has an obvious movement or deformation during the PET scan, and motion correction is needed for the PET images or the preliminary PET parametric image; if the similarity is not smaller than the similarity threshold, the processing device 140 may determine that the target subject has no obvious movement or deformation during the PET scan, and motion correction is not needed for the PET images or the preliminary PET parametric image.

According to the process 900, since the motion of the target subject during the PET scan directly affects the similarity between the lesion segmentation results corresponding different PET parameters (e.g., the first lesion segmentation result and the second lesion segmentation result). Therefore, the second motion evaluation result obtained based on the similarity may have a relatively large accuracy.

FIG. 9 is a flowchart illustrating an exemplary process 900 for determining whether noise correction is needed according to some embodiments of the present disclosure. In some embodiments, one or more operations of the process 900 may be performed to achieve at least part of operation 420 as described in connection with FIG. 4.

In 910, for each physical point of the target subject, the processing device 140 (e.g., the determination module 320) may determine a TAC of the physical point based on pixel values corresponding to the physical point in the PET images.

As used herein, a TAC of a physical point refers to a curve that represents a change in a concentration of the tracer over time at the physical point. In some embodiments, the TAC may include a measurement TAC and/or a fitting TAC.

Specifically, for each PET image, the processing device 140 may determine a time period during the PET scan for collecting the PET data that is used to reconstruct the PET image. For each PET image, the processing device 140 may determine a standard uptake value (SUV) of the physical point in the PET image. Further, the processing device 140 may generate the TAC of the physical point based on the time periods and the SUVs corresponding to the PET images. For example, the processing device 140 may determine points in a coordinate system based on the time periods and the SUVs corresponding to the PET images, wherein the coordinate system has the time and the SUV as two coordinate axes. Coordinates of a point include a time and the SUV corresponding to one PET image. Then, the processing device 140 may generate the fitting TAC of the physical point by fitting the points in the coordinate system using a mathematic fitting algorithm. Alternatively, the processing device 140 may generate the measurement TAC of the physical point by connecting the points in the coordinate system. In some embodiments, the fitting TAC may be determined based on a pharmacokinetic model for determining a PET parametric value of the physical point. Specifically, the processing device 140 may input the pixel values corresponding to the physical point in the PET images into the pharmacokinetic model, and the pharmacokinetic model may output the second TAC.

In 920, for each physical point of the target subject, the processing device 140 (e.g., the determination module 320) may determine a noise level parameter corresponding to the physical point based on the TAC of the physical point.

In some embodiments, the processing device 140 may obtain a noise evaluation model from a storage device (e.g., the storage device 150 or an external source). Further, the processing device 140 may determine the noise level parameter corresponding to the physical point based on the TAC of the physical point using the noise evaluation model. In some embodiments, the noise evaluation model may include a Gaussian denoising model. Specifically, the processing device 140 may input the TAC of the physical point into the noise evaluation model, and the noise evaluation model may output the noise level parameter corresponding to the physical point.

For example, the TAC may include both the measurement TAC and the fitting TAC, and the noise evaluation model may determine the noise level parameter corresponding to the physical point according to the Equation (1) as blow:

$${α}^{2} = \frac{1}{{N}_{frame} - {N}_{parameters}} {\sum }_{i = 1}^{{N}_{frame}} \frac{{( - {y}_{i} )}^{2}}{{e}^{λ t} i / Δ {t}_{i}} , \qquad ( 1 )$$

where, N_{frame} denotes a count of the PET images, Nₚₐᵣₐₘₑₜₑᵣₛ denotes a count of PET parameters of the pharmacokinetic model corresponding to the physical point, yᵢ denotes the measurement TAC of the physical point, [symbol] denotes the fitting TAC of the physical point, λ denotes decay constant, t; denotes the time period corresponding to the i^{th} PET image, Δtᵢ denotes a duration of the time period corresponding to the i^{th} PET image, i is a positive integer greater than or equal to 1 and smaller than or equal to N_{frame}.

In 930, the processing device 140 (e.g., the determination module 320) may determine whether noise correction is needed based on the noise level parameter corresponding to each physical point.

The processing device 140 may determine an average of the noise level parameters corresponding to the physical points, and designate the average as a total noise level parameter of the PET images. In response to determining that the total noise level parameter is greater than a noise level threshold, the processing device 140 may determine that the noise level of the PET images is relatively large, and noise correction is needed for the PET images. In response to determining that the total noise level parameter is not greater than the noise level threshold, the processing device 140 may determine that the noise level of the PET images is relatively small, and noise correction is not needed for the PET images.

FIG. 10 is a flowchart illustrating an exemplary process 1000 for determining whether pharmacokinetic model correction is needed according to some embodiments of the present disclosure. The process 1000 may be performed when the same pharmacokinetic model is used for each physical point when generating the preliminary PET parametric image. In some embodiments, one or more operations of the process 1000 may be performed to achieve at least part of operation 420 as described in connection with FIG. 4.

In 1010, for each physical point of the target subject, the processing device 140 (e.g., the determination module 320) may determine a matching degree of the pharmacokinetic model with respect to the physical point based on the corrected PET images or the PET images.

In some embodiments, a matching degree of a pharmacokinetic model with respect to a physical point may reflect an adaptability of the pharmacokinetic model to the physical point when the preliminary PET parametric image is generated using the pharmacokinetic model.

As described in operation 420, the preliminary PET parametric image refers to a preliminarily determined PET parametric image generated by processing the corrected PET images described in operations 440 and 450 or the PET images using at least one pharmacokinetic model. If the preliminarily PET parametric image is generated by processing the corrected PET images, for each physical point of the target subject, the processing device 140 may determine the matching degree of the pharmacokinetic model with respect to the physical point based on the corrected PET images. If the preliminarily PET parametric image is generated by processing the PET images, for each physical point of the target subject, the processing device 140 may determine the matching degree of the pharmacokinetic model with respect to the physical point based on the PET images.

For illustration purposes, the preliminary PET parametric image generated by processing the PET images is described hereinafter.

In some embodiments, the processing device 140 may determine a first TAC of the physical point based on pixel values corresponding to the physical point in the PET images. The first TAC of the physical point may be a measurement TAC. More descriptions of the generation manner of the measurement TAC can be found in descriptions of operation 710. Further, the processing device 140 may determine a second TAC of the physical point based on the pixel values corresponding to the physical point in the PET images and the pharmacokinetic model corresponding to the physical point. The second TAC may be a fitting TAC determined by the pharmacokinetic model. Specifically, the processing device 140 may input the pixel values corresponding to the physical point in the PET images into the pharmacokinetic model, and the pharmacokinetic model may output the second TAC.

Then, the processing device 140 may determine the matching degree based on the first TAC and the second TAC. Specifically, the processing device 140 may determine a similarity between the first TAC and the second TAC. The similarity between the first TAC and the second TAC may be represented by such as a Akaike information criterion (AIC) parameter. In some embodiments, the processing device 140 may determine the similarity between the first TAC and the second TAC using a similarity algorithm (e.g., the similarity algorithms described in operation 930). Further, the processing device 140 may determine the matching degree based on the similarity between the first TAC and the second TAC. The greater the similarity between the first TAC and the second TAC is, the greater the matching degree may be.

In 1020, for each physical point of the target subject, the processing device 140 (e.g., the determination module 320) may generate a determination result indicating whether the corresponding pixel of the physical point in the preliminary PET parametric image needs to be corrected based on the matching degree.

In response to determining that the matching degree corresponding to the physical point is smaller than a matching degree threshold, the processing device 140 may generate the determination result indicating that the corresponding pixel of the physical point in the preliminary PET parametric image needs to be corrected; in response to determining that the matching degree corresponding to the physical point is not smaller than the matching degree threshold, the processing device 140 may generate the determination result indicating that the corresponding pixel of the physical point in the preliminary PET parametric image does not need to be corrected.

In 1030, the processing device 140 (e.g., the determination module 320) may determine whether pharmacokinetic correction is needed based on the determination result corresponding to each physical point.

In some embodiments, if there are one or more physical points whose pixels in the preliminary PET parametric image needs to be corrected, the processing device 140 may determine that the pharmacokinetic model for generating the preliminary PET parametric image does not satisfy requirements, and pharmacokinetic correction is needed; if there is no physical point whose pixel in the preliminary PET parametric image needs to be corrected, the processing device 140 may determine that the pharmacokinetic model for generating the preliminary PET parametric image satisfies requirements, and pharmacokinetic correction is not needed.

In some embodiments, if a count of the physical points whose pixels in the preliminary PET parametric image needs to be corrected is greater than a count threshold, the processing device 140 may determine that the pharmacokinetic model for generating the preliminary PET parametric image does not satisfy requirements, and pharmacokinetic correction is needed; if the count of the physical points whose pixels in the preliminary PET parametric image needs to be corrected is not greater than the count threshold, the processing device 140 may determine that the pharmacokinetic model for generating the preliminary PET parametric image satisfies requirements, and pharmacokinetic correction is not needed.

According to process 1000, since the fitting TAC (i.e., the second TAC) is determined using the pharmacokinetic model corresponding to the physical point, the matching degree of the pharmacokinetic model with respect to the physical point determined based on the measurement TAC and the fitting TAC may be relatively accurate. Therefore, the pharmacokinetic model evaluation result based on the matching degree corresponding to each physical point may be relatively accurate.

It should be noted that the processes 400-600 and 800-1000 and the descriptions thereof are provided for the purposes of illustration, and not intended to limit the scope of the present disclosure. For persons having ordinary skills in the art, various modifications and changes in the forms and details of the application of the above method and system may occur without departing from the principles of the present disclosure. However, those variations and modifications also fall within the scope of the present disclosure. For example, the operations of the illustrated processes 400-600 and 800-1000 are intended to be illustrative. In some embodiments, the processes 400-600 and 800-1000 may be accomplished with one or more additional operations not described, and/or without one or more of the operations discussed. Additionally, the order in which the operations of the processes 400-600 and 800-1000 and regarding descriptions are not intended to be limiting.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and “some embodiments” mean that a particular feature, structure or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures or characteristics may be combined as suitable in one or more embodiments of the present disclosure.

Further, it will be appreciated by one skilled in the art, aspects of the present disclosure may be illustrated and described herein in any of a number of patentable classes or context including any new and useful process, machine, manufacture, or composition of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “module,” “unit,” “component,” “device,” or “system.” Furthermore, aspects of the present disclosure may take the form of a computer program product embodied in one or more computer readable media having computer readable program code embodied thereon.

A computer readable signal medium may include a propagated data signal with computer readable program code embodied therein, for example, in baseband or as part of a carrier wave. Such a propagated signal may take any of a variety of forms, including electro-magnetic, optical, or the like, or any suitable combination thereof. A computer readable signal medium may be any computer readable medium that is not a computer readable storage medium and that may communicate, propagate, or transport a program for use by or in connection with an instruction execution system, apparatus, or device. Program code embodied on a computer readable signal medium may be transmitted using any appropriate medium, including wireless, wireline, optical fiber cable, RF, or the like, or any suitable combination of the foregoing.

Computer program code for carrying out operations for aspects of the present disclosure may be written in any combination of one or more programming languages, including an subject oriented programming language such as Java, Scala, Smalltalk, Eiffel, JADE, Emerald, C++, C#, VB. NET, Python or the like, conventional procedural programming languages, such as the “C” programming language, Visual Basic, Fortran 2003, Perl, COBOL 2002, PHP, ABAP, dynamic programming languages such as Python, Ruby and Groovy, or other programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider) or in a cloud computing environment or offered as a service such as a Software as a Service (SaaS).

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. This method of disclosure, however, is not to be interpreted as reflecting an intention that the claimed subject matter requires more features than are expressly recited in each claim. Rather, claim subject matter lie in less than all features of a single foregoing disclosed embodiment.

In some embodiments, the numbers expressing quantities or properties used to describe and claim certain embodiments of the application are to be understood as being modified in some instances by the term “about,” “approximate,” or “substantially.” For example, “about,” “approximate,” or “substantially” may indicate a certain variation (e.g., ±1%, ±5%, ±10%, or ±20%) of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable. In some embodiments, a classification condition used in classification or determination is provided for illustration purposes and modified according to different situations. For example, a classification condition that “a value is greater than the threshold value” may further include or exclude a condition that “the probability value is equal to the threshold value.”

What is claimed is:

1. A method for parametric imaging in positron emission tomography (PET), implemented on a computing device having at least one processor and at least one storage device, the method comprising:
obtaining multiple PET images collected via a PET scan of a target subject, the PET images being dynamic PET images for difference time periods during the PET scan and used to generate a target PET parametric image of the target subject;
determining whether data correction needs to be performed in a process of generating the target PET parametric image; and
in response to determining that data correction needs to be performed, correcting the PET images or a preliminary PET parametric image, and generating the target PET parametric image of the target subject based on the corrected PET images or the corrected preliminary PET parametric image, wherein the preliminary PET parametric image is generated by processing the corrected PET images or the PET images using at least one pharmacokinetic model.

2. The method of claim 1, wherein the determining whether data correction needs to be performed in a process of generating the target PET parametric image comprises:
for each of the PET images, generating an organ segmentation image by segmenting organs in the PET image;
determining motion information of the organs of the target subject based on the organ segmentation image corresponding to each of the PET images;
determining whether motion correction needs to be performed in the process of generating the target PET parametric based on the motion information of the organs.

3. The method of claim 1, wherein the determining whether data correction needs to be performed in a process of generating the target PET parametric image comprises:
for each of the PET images,
determining, in the PET image, a target region corresponding to a target organ;
determining a variation of pixel values in the target region; and
determining whether noise correction needs to be performed in the process of generating the target PET parametric image based on the variation corresponding to each of the PET images.

4. The method of claim 1, wherein the determining whether data correction needs to be performed in a process of generating the target PET parametric image comprises:
for each physical point of the target subject,
determining a pharmacokinetic model corresponding to the physical point; and
determining a PET parametric value of the physical point based on pixel values corresponding to the physical point in the corrected PET images or the PET images using the pharmacokinetic model of the physical point;
generating the preliminary PET parametric image based on the PET parametric value of each physical point of the target subject; and
determining whether data correction needs to be performed in the process of generating the target PET parametric image based on the preliminary PET parametric image.

5. The method of claim 4, wherein the preliminary PET parametric image includes a first preliminary PET parametric image and a second preliminary PET parametric image corresponding to different PET parameters, and the determining whether data correction needs to be performed in the process of generating the target PET parametric image based on the preliminary PET parametric image comprises:
determining a first lesion segmentation result by segmenting lesion areas from the first preliminary PET parametric image;
determining a second lesion segmentation result by segmenting lesion areas from the second preliminary PET parametric image;
determining a similarity between the first lesion segmentation result and the second lesion segmentation result; and
determining whether motion correction needs to be performed in the process of generating the target PET parametric image based on the similarity.

6. The method of claim 4, wherein the determining whether data correction needs to be performed in a process of generating the target PET parametric image comprises:
for each physical point of the target subject,
determining a time-activity curve (TAC) of the physical point based on pixel values corresponding to the physical point in the PET images;
determining a noise level parameter corresponding to the physical point based on the TAC of the physical point; and
determining whether noise correction needs to be performed in a process of generating the target PET parametric image based on the noise level parameter corresponding to each physical point.

7. The method of claim 4, wherein the determining whether data correction needs to be performed in the process of generating the target PET parametric image based on the preliminary PET parametric image comprises:
for each physical point of the target subject,
determining a matching degree of the pharmacokinetic model with respect to the physical point based on the corrected PET images or the PET images;
generating a determination result indicating whether the corresponding pixel of the physical point in the preliminary PET parametric image needs to be corrected based on the matching degree;
determining whether pharmacokinetic model correction needs to be performed in the process of generating the target PET parametric image based on the determination result corresponding to each physical point.

8. The method of claim 4, wherein the determining a pharmacokinetic model corresponding to the physical point comprises:
determining a TAC of the physical point based on pixel values corresponding to the physical point in the PET images;
selecting, from multiple candidate pharmacokinetic models, the pharmacokinetic model corresponding to the physical point based on the TAC of the physical point.

9. The method of claim 8, wherein the pharmacokinetic model corresponding to the physical point is determined by processing the TAC of the physical point using a selection model, the selection model being generated by training a preliminary model using training samples, each of the training samples corresponding to a sample physical point and being determined by:
determining a sample TAC of the sample physical point based on sample PET images collected in a sample PET scan of a sample subject;
determining predicted TACs of the sample physical point corresponding to the candidate pharmacokinetic models by processing the sample PET images using the candidate pharmacokinetic models;
determining a recommended pharmacokinetic model from the candidate pharmacokinetic models based on the sample TAC and the predicted TACs;
designating the sample TAC as a training input of the training sample and the recommended pharmacokinetic model as a training label of the training sample.

10. The method of claim 1, wherein the determining whether data correction needs to be performed in a process of generating the target PET parametric image comprises:
obtaining sets of PET raw data collected in the PET scan, the PET images being reconstructed from the sets of PET raw data;
for each set of PET raw data, generating a histoimage corresponding to the set of PET raw data;
determining motion information of organs of the target subject based on the histoimage corresponding to each set of PET raw data; and
determining whether motion correction needs to be performed in the process of generating the target PET parametric image based on the motion information of the organs.

11. The method of claim 1, wherein the determining whether data correction needs to be performed in a process of generating the target PET parametric image comprises:
obtaining sets of PET raw data collected in the PET scan, the PET images being reconstructed from the sets of PET raw data;
for each set of PET raw data,
determining a coincidence event count based on the set of PET raw data;
determining a noise level parameter of the set of PET raw data based on the coincidence event count corresponding to the set of PET raw data;
determining whether noise correction needs to be performed in the process of generating the target PET parametric image based on the noise level parameter of each set of PET raw data.

12. The method of claim 1, wherein the obtaining multiple PET images collected in a PET scan of the target subject comprises:
obtaining multiple sets of PET raw data collected in the PET scan;
for each set of PET raw data,
determining coincidence event count based on the set of PET raw data;
determining a reconstruction algorithm for reconstructing the set of PET raw data based on the coincidence event count; and
generating the PET images by reconstructing the sets of PET raw data using their respective reconstruction algorithms.

13. The method of claim 1, wherein the determining whether data correction needs to be performed in a process of generating the target PET parametric image comprises:
performing a first quality evaluation on the PET images;
performing a second quality evaluation on the preliminary PET parametric image;
determining whether data correction needs to be performed in the process of generating the target PET parametric image based on results of the first quality evaluation and the second quality evaluation.

14. A system for parametric imaging in positron emission tomography (PET), comprising:
at least one storage device including a set of instructions; and
at least one processor in communication with the at least one storage device, wherein when executing the set of instructions, the at least one processor is configured to direct the system to perform operations including:
obtaining multiple PET images collected via a PET scan of a target subject, the PET images being dynamic PET images for difference time periods during the PET scan and used to generate a target PET parametric image of the target subject;
determining whether data correction needs to be performed in a process of generating the target PET parametric image; and
in response to determining that data correction needs to be performed, correcting the PET images or a preliminary PET parametric image, and generating the target PET parametric image of the target subject based on the corrected PET images or the corrected preliminary PET parametric image, wherein the preliminary PET parametric image is generated by processing the corrected PET images or the PET images using at least one pharmacokinetic model.

15. The system of claim 14, wherein the determining whether data correction needs to be performed in a process of generating the target PET parametric image comprises:
for each of the PET images, generating an organ segmentation image by segmenting organs in the PET image;
determining motion information of the organs of the target subject based on the organ segmentation image corresponding to each of the PET images;
determining whether motion correction needs to be performed in the process of generating the target PET parametric based on the motion information of the organs.

16. The system of claim 14, wherein the determining whether data correction needs to be performed in a process of generating the target PET parametric image comprises:
for each of the PET images,
determining, in the PET image, a target region corresponding to a target organ;
determining a variation of pixel values in the target region; and
determining whether noise correction needs to be performed in the process of generating the target PET parametric image based on the variation corresponding to each of the PET images.

17. The system of claim 14, wherein the determining whether data correction needs to be performed in a process of generating the target PET parametric image comprises:
for each physical point of the target subject,
determining a pharmacokinetic model corresponding to the physical point; and
determining a PET parametric value of the physical point based on pixel values corresponding to the physical point in the corrected PET images or the PET images using the pharmacokinetic model of the physical point;
generating the preliminary PET parametric image based on the PET parametric value of each physical point of the target subject; and
determining whether data correction needs to be performed in the process of generating the target PET parametric image based on the preliminary PET parametric image.

18. The system of claim 17, wherein the preliminary PET parametric image includes a first preliminary PET parametric image and a second preliminary PET parametric image corresponding to different PET parameters, and the determining whether data correction needs to be performed in the process of generating the target PET parametric image based on the preliminary PET parametric image comprises:
determining a first lesion segmentation result by segmenting lesion areas from the first preliminary PET parametric image;
determining a second lesion segmentation result by segmenting lesion areas from the second preliminary PET parametric image;
determining a similarity between the first lesion segmentation result and the second lesion segmentation result; and
determining whether motion correction needs to be performed in the process of generating the target PET parametric image based on the similarity.

19. The system of claim 17, wherein the determining whether data correction needs to be performed in a process of generating the target PET parametric image comprises:
for each physical point of the target subject,
determining a time-activity curve (TAC) of the physical point based on pixel values corresponding to the physical point in the PET images;
determining a noise level parameter corresponding to the physical point based on the TAC of the physical point; and
determining whether noise correction needs to be performed in a process of generating the target PET parametric image based on the noise level parameter corresponding to each physical point.

20. A non-transitory computer readable medium, comprising at least one set of instructions for parametric imaging in positron emission tomography (PET), wherein when executed by one or more processors of a computing device, the at least one set of instructions causes the computing device to perform a method, the method comprising:
obtaining multiple PET images collected via a PET scan of a target subject, the PET images being dynamic PET images for difference time periods during the PET scan and used to generate a target PET parametric image of the target subject;
determining whether data correction needs to be performed in a process of generating the target PET parametric image; and
in response to determining that data correction needs to be performed, correcting the PET images or a preliminary PET parametric image, and generating the target PET parametric image of the target subject based on the corrected PET images or the corrected preliminary PET parametric image, wherein the preliminary PET parametric image is generated by processing the corrected PET images or the PET images using at least one pharmacokinetic model.
